(19) United States
(12) Patent Application Publication
(10) Pub. No.: US 2024/0285231 A1
(43) Pub. Date: Aug. 29, 2024

(54) System and Method for Determining a Stool Condition

(71) Applicant: Dieta Inc., Oak Park, CA (US)

(72) Inventors: Asaf Kraus, Oak Park, CA (US); Austin Mckay, Oak Park, CA (US); Benjamin Neigher, Oak Park, CA (US)

(21) Appl. No.: 18/572,148

(22) PCT Filed: Jun. 17, 2022
(86) PCT No.: PCT/US22/34097

§ 371 Date: Dec. 19, 2023

Related U.S. Application Data
(60) Provisional application No. 63/212,708, filed on Jun. 20, 2021.
(60) Provisional application No. 63/231,349, filed on Aug. 10, 2021.

(51) Int. Cl. A61B 5/00; G06T 7/00; G16H 30/20; G16H 50/30

(52) U.S. Cl. CPC A61B 5/4842; G06T 7/0012; G16H 30/20; G16H 50/30

(57) ABSTRACT

Disclosed herein, in some aspects, are systems and methods for determining and/or monitoring a stool condition for a subject. In some embodiments, the stool condition is based on one or more images of stool of a subject. In some embodiments, the stool condition correlates with a stool assessment comprising i) a characterization of the stool according to a plurality of characteristics, and/or ii) identifying one or more medical conditions, illnesses, and/or diseases associated with the stool. In some embodiments, the stool condition is determined using one or more Artificial Intelligence engines using a trained data set. In some embodiments, the stool condition is based on one or more stool assessments performed for one or more stools corresponding to one or more bowel movements over a period of time.

### CROSS REFERENCE TO RELATED APPLICATIONS

This PCT application claims priority to U.S. Provisional Application No. 63/212,708, filed Jun. 20, 2021; and U.S. Provisional Application No. 63/231,349, filed Aug. 10, 2021, the contents of each which are incorporated herein by reference in its entirety.

### BACKGROUND

Stool samples can be indicative of health conditions in a subject. Chemical analysis of stool may provide intrinsic information relating to gut health, for example. By contrast, the visual appearance of the stool may be indicative of a condition relating to the movement of bowels, such as identifying a subject being constipated or having diarrhea. Other visual indicators of stool may provide further indicative measures of a bowel movement condition. Such self-assessed visual inspection of stool, however, is often subjective and open to inconsistencies for periodic evaluation. Therefore, there is a need for a more robust visual evaluation of stool.

### SUMMARY

Disclosed herein in some aspects is a non-transitory computer readable medium for determining a stool condition for a subject, the non-transitory computer readable medium comprising instructions that, when executed by a processor, cause the processor to perform operations including: a) receiving an image of stool corresponding to a bowel movement; b) determining a plurality of characteristics associated with the stool based on the image; and c) performing a stool assessment based on the plurality of characteristics, the stool assessment correlating with the stool condition; wherein the plurality of characteristics comprises one or more of a shape and texture, consistency, fragmentation, fuzziness, and volume.

In some embodiments, the stool condition is based on a plurality of images of stools corresponding to a plurality of bowel movements, wherein a stool assessment is performed for each image of stool. In some embodiments, performing the stool assessment further comprises identifying one or more medical conditions, illnesses, and/or diseases for the subject. In some embodiments, the one or more medical conditions, illnesses, and/or diseases comprises Irritable Bowel Syndrome, Crohn's Disease, Ulcerative Colitis, Hepatic Encephalopathy, or a combination thereof. In some embodiments, the operations further include identifying one or more correlations between one or more subject conditions and the stool condition. In some embodiments, the one or more subject conditions comprises a diet intake, one or more lifestyle conditions, one or more medications, or a combination thereof.

In some embodiments, the operations further includes determining an effectiveness of a medication based on a change in the stool condition between one or more bowel movements. In some embodiments, the operations further includes providing an intervention recommendation based on the stool condition. In some embodiments, the recommendation comprises a change to i) one or more of the subject's diet, ii) one or more lifestyle conditions, and/or iii) one or more medications being received by the subject.

In some embodiments, determining the plurality of characteristics and/or performing the stool assessment comprises using a machine learning algorithm. In some embodiments, the machine learning algorithm uses a trained data set in operative communication with the processor to determine the plurality of characteristics and/or perform the stool assessment. In some embodiments, the trained data set comprises a plurality of past images of stool correlated with a plurality of characteristics.

In some embodiments, the processor is a part of computing device. In some embodiments, the computing device comprises a mobile device, a desktop, a laptop, and/or a remote computing server. In some embodiments, the mobile device comprises a smart phone, a tablet, a smartwatch, or any combination thereof. In some embodiments, receiving the image of the stool comprises using a camera in operative communication with the processor and configured to capture the image. In some embodiments, the computing device comprises said camera. In some embodiments, the computing device is in operative communication with a display to output the stool assessment. In some embodiments, the display is in operative communication with the camera, such that the display provides guiding features to capture the image. In some embodiments, the guiding features comprises a shape of a toilet seat defining a central area, such that the image of the stool is located within the central area when the image is captured.

In some embodiments, the stool assessment comprises a score and/or rating relating to each characteristic of the plurality of characteristics. In some embodiments, the plurality of characteristics comprises consistency, wherein the corresponding score and/or rating corresponds to a liquid to solid scale of the stool, wherein one end of the scale corresponds to a fully liquid stool, and another end of the scale corresponds to a fully solid stool. In some embodiments, the plurality of characteristics comprises fragmentation, wherein the corresponding score and/or rating corresponds to a degree relating to a number of pieces present in the stool, wherein one end of the scale corresponds to a single stool piece, and another end of the scale corresponds to a large number of stool pieces. In some embodiments, the plurality of characteristics comprises fuzziness, wherein the corresponding score and/or rating corresponds to a degree of a clear boundary existing between the stool and a background in the image, wherein one end of the scale corresponds to a clear distinguishable or substantially distinguishable boundary, and another end of the scale corresponds to an indistinguishable or substantially indistinguishable boundary. In some embodiments, the plurality of characteristics comprises volume, wherein the corresponding score and/or rating corresponds to a size of the stool, wherein one end of the scale corresponds to a small size, and another end of the scale corresponds to a large size stool.

In some embodiments, the operations further include i) sending to the stool assessment to a healthcare provider, and/or ii) receiving input from the healthcare provider.

In some embodiments, the processor is in operative communication with the healthcare provider via a communication module. In some embodiments, obtaining an image comprises obtaining a plurality of images of the stool, wherein determining the plurality of characteristics and outputting the stool assessment is based on the plurality of images.

In some embodiments, the operations further comprises validating the stool assessment performed based on comparing a score for one or more of the plurality of characteristics between i) the image and one or more other images of the stool, and/or ii) the stool assessment and one or more other stool assessments performed for the image.

Disclosed herein, in some aspects, is a method for determining a stool condition for a subject, the method comprising: a) receiving an image of stool corresponding to a bowel movement; b) determining a plurality of characteristics associated with the stool based on the image; and c) performing a stool assessment based on the plurality of characteristics, the stool assessment correlating with the stool condition; wherein the plurality of characteristics comprises one or more of a shape and texture, consistency, fragmentation, fuzziness, and volume.

In some embodiments, the stool condition is based on a plurality of images of stools corresponding to a plurality of bowel movements, wherein a stool assessment is performed for each image of stool. In some embodiments, performing the stool assessment further comprises identifying one or more medical conditions, illnesses, and/or diseases for the subject. In some embodiments, the one or more medical conditions, illnesses, and/or diseases comprises Irritable Bowel Syndrome, Crohn's Disease, Ulcerative Colitis, Hepatic Encephalopathy, or a combination thereof. In some embodiments, the method further comprises identifying one or more correlations between one or more subject conditions and the stool condition. In some embodiments, the one or more subject conditions comprises a diet intake, one or more lifestyle conditions, one or more medications, or a combination thereof.

In some embodiments, the method further comprises determining an effectiveness of a medication based on a change in the stool condition between one or more bowel movements.

In some embodiments, the method further comprises providing an intervention recommendation based on the stool condition. In some embodiments, the recommendation comprises a change to i) one or more of the subject's diet, ii) one or more lifestyle conditions, and/or iii) one or more medications being received by the subject.

In some embodiments, determining the plurality of characteristics and/or performing the stool assessment comprises using a machine learning algorithm. In some embodiments, the machine learning algorithm uses a trained data set in operative communication with the processor to determine the plurality of characteristics and/or perform the stool assessment. In some embodiments, the trained data set comprises a plurality of past images of stool correlated with a plurality of characteristics.

In some embodiments, the processor is a part of computing device. In some embodiments, the computing device comprises a mobile device, a desktop, a laptop, and/or a remote computing server. In some embodiments, the mobile device comprises a smart phone, a tablet, a smartwatch, or any combination thereof. In some embodiments, receiving the image of the stool comprises using a camera in operative communication with the processor and configured to capture the image. In some embodiments, the computing device comprises said camera. In some embodiments, the computing device is in operative communication with a display to output the stool assessment. In some embodiments, the display is in operative communication with the camera, such that the display provides guiding features to capture the image. In some embodiments, the guiding features comprises a shape of a toilet seat defining a central area, such that the image of the stool is located within the central area when the image is captured.

In some embodiments, the stool assessment comprises a score and/or rating relating to each characteristic of the plurality of characteristics. In some embodiments, the plurality of characteristics comprises consistency, wherein the corresponding score and/or rating corresponds to a liquid to solid scale of the stool, wherein one end of the scale corresponds to a fully liquid stool, and another end of the scale corresponds to a fully solid stool. In some embodiments, the plurality of characteristics comprises fragmentation, wherein the corresponding score and/or rating corresponds to a degree relating to a number of pieces present in the stool, wherein one end of the scale corresponds to a single stool piece, and another end of the scale corresponds to a large number of stool pieces. In some embodiments, the plurality of characteristics comprises fuzziness, wherein the corresponding score and/or rating corresponds to a degree of a clear boundary existing between the stool and a background in the image, wherein one end of the scale corresponds to a clear distinguishable or substantially distinguishable boundary, and another end of the scale corresponds to an indistinguishable or substantially indistinguishable boundary. In some embodiments, the plurality of characteristics comprises volume, wherein the corresponding score and/or rating corresponds to a size of the stool, wherein one end of the scale corresponds to a small size, and another end of the scale corresponds to a large size stool.

In some embodiments, the method further comprises i) sending to the stool assessment to a healthcare provider, and/or ii) receiving input from the healthcare provider.

In some embodiments, the processor is in operative communication with the healthcare provider via a communication module. In some embodiments, obtaining an image comprises obtaining a plurality of images of the stool, wherein determining the plurality of characteristics and outputting the stool assessment is based on the plurality of images.

In some embodiments, the method further comprises validating the stool assessment performed based on comparing a score for one or more of the plurality of characteristics between i) the image and one or more other images of the stool, and/or ii) the stool assessment and one or more other stool assessments performed for the image.

Disclosed herein, in some aspects, is a system for determining a stool condition for a subject, the system comprising: a) one or more processors; and b) one or more memories storing instructions that, when executed by the one or more processors, cause the system to perform operations including: i) receiving an image of stool corresponding to a bowel movement; ii) determining a plurality of characteristics associated with the stool based on the image; and iii) performing a stool assessment based on the plurality of characteristics, the stool assessment correlating with the stool condition; wherein the plurality of characteristics comprises one or more of a shape and texture, consistency, fragmentation, fuzziness, and volume.

In some embodiments, the stool condition is based on a plurality of images of stools corresponding to a plurality of bowel movements, wherein a stool assessment is performed for each image of stool. In some embodiments, performing the stool assessment further comprises identifying one or more medical conditions, illnesses, and/or diseases for the subject. In some embodiments, the one or more medical conditions, illnesses, and/or diseases comprises Irritable Bowel Syndrome, Crohn's Disease, Ulcerative Colitis, Hepatic Encephalopathy, or a combination thereof. In some embodiments, the operations further include identifying one or more correlations between one or more subject conditions and the stool condition. In some embodiments, the one or more subject conditions comprises a diet intake, one or more lifestyle conditions, one or more medications, or a combination thereof.

In some embodiments, the operations further includes determining an effectiveness of a medication based on a change in the stool condition between one or more bowel movements. In some embodiments, the operations further includes providing an intervention recommendation based on the stool condition. In some embodiments, the recommendation comprises a change to i) one or more of the subject's diet, ii) one or more lifestyle conditions, and/or iii) one or more medications being received by the subject.

In some embodiments, determining the plurality of characteristics and/or performing the stool assessment comprises using a machine learning algorithm. In some embodiments, the machine learning algorithm uses a trained data set in operative communication with the processor to determine the plurality of characteristics and/or perform the stool assessment. In some embodiments, the trained data set comprises a plurality of past images of stool correlated with a plurality of characteristics.

In some embodiments, the processor is a part of computing device. In some embodiments, the computing device comprises a mobile device, a desktop, a laptop, and/or a remote computing server. In some embodiments, the mobile device comprises a smart phone, a tablet, a smartwatch, or any combination thereof. In some embodiments, receiving the image of the stool comprises using a camera in operative communication with the processor and configured to capture the image. In some embodiments, the computing device comprises said camera. In some embodiments, the computing device is in operative communication with a display to output the stool assessment. In some embodiments, the display is in operative communication with the camera, such that the display provides guiding features to capture the image. In some embodiments, the guiding features comprises a shape of a toilet seat defining a central area, such that the image of the stool is located within the central area when the image is captured.

In some embodiments, the stool assessment comprises a score and/or rating relating to each characteristic of the plurality of characteristics. In some embodiments, the plurality of characteristics comprises consistency, wherein the corresponding score and/or rating corresponds to a liquid to solid scale of the stool, wherein one end of the scale corresponds to a fully liquid stool, and another end of the scale corresponds to a fully solid stool. In some embodiments, the plurality of characteristics comprises fragmentation, wherein the corresponding score and/or rating corresponds to a degree relating to a number of pieces present in the stool, wherein one end of the scale corresponds to a single stool piece, and another end of the scale corresponds to a large number of stool pieces. In some embodiments, the plurality of characteristics comprises fuzziness, wherein the corresponding score and/or rating corresponds to a degree of a clear boundary existing between the stool and a background in the image, wherein one end of the scale corresponds to a clear distinguishable or substantially distinguishable boundary, and another end of the scale corresponds to an indistinguishable or substantially indistinguishable boundary. In some embodiments, the plurality of characteristics comprises volume, wherein the corresponding score and/or rating corresponds to a size of the stool, wherein one end of the scale corresponds to a small size, and another end of the scale corresponds to a large size stool.

In some embodiments, the operations further include i) sending to the stool assessment to a healthcare provider, and/or ii) receiving input from the healthcare provider.

In some embodiments, the processor is in operative communication with the healthcare provider via a communication module. In some embodiments, obtaining an image comprises obtaining a plurality of images of the stool, wherein determining the plurality of characteristics and outputting the stool assessment is based on the plurality of images.

In some embodiments, the operations further comprises validating the stool assessment performed based on comparing a score for one or more of the plurality of characteristics between i) the image and one or more other images of the stool, and/or ii) the stool assessment and one or more other stool assessments performed for the image.

### BRIEF DESCRIPTION OF THE DRAWINGS

These and other features, aspects, and advantages of some embodiments will become better understood with regard to the following description and accompanying drawings.

FIG. 1 depicts a system environment overview for determining a stool condition, in accordance with an embodiment.

FIG. 2 depicts a block diagram of the stool evaluation tool, in accordance with an embodiment.

FIG. 3 depicts an exemplary flow chart for determining a stool condition, in accordance with an embodiment.

FIG. 4 depicts an exemplary computer system, in accordance with an embodiment.

FIG. 5 depicts exemplary categories for the shape and texture characteristic of a stool, here with reference to the Bristol Stool Scale, in accordance with an embodiment.

FIG. 6 depicts exemplary depiction of a display for a computing device in operative communication with an image capture device, depicting guiding features for capturing an image of a stool, in accordance with an embodiment.

FIG. 7 depicts exemplary depiction of a display for a computing device showing an output of a determined stool condition, in accordance with an embodiment.

FIG. 8 depicts exemplary depiction of a display for a computing device showing an output of multiple determined stool conditions, in accordance with an embodiment.

FIG. 9 depicts exemplary depiction of a display for a computing device showing an output of the multiple determined stool conditions along with a summary of symptoms, and prompts for additional modules, in accordance with an embodiment.

FIG. 10 depicts exemplary depiction of a system for determining a stool condition, in accordance with an embodiment.

FIGS. 11A-E depicts exemplary images of stool at different increments for the plurality of characteristics, in accordance with an embodiment.

FIG. 12 depicts exemplary depiction of a display for a computing device showing various modules of a stool evaluation tool, in accordance with an embodiment.

FIGS. 13A-13B depict exemplary data corresponding to an experiment for determinant an efficacy of a medication with respect to a stool condition, in accordance with an embodiment.

### DETAILED DESCRIPTION

### I. Definitions

Terms used in the claims and specification are defined as set forth below unless otherwise specified.

The terms “subject” or “patient” are used interchangeably and encompass a cell, tissue, or organism, human or non-human, whether in vivo, ex vivo, or in vitro, male or female.

The terms “treating,” “treatment,” or “therapy” may be used interchangeably.

The terms “stool”, “stool sample”, or “feces” may be used interchangeably. The term stool refers to stool (feces) expelled by a subject during a bowel movement session. The stool is the total stool expelled during the bowel movement session (regardless of number of pieces, texture, liquid/solid ratio, etc.).

The term “bowel movement” or “bowel movement session” may be used interchangeably. The term bowel movement refers to a passing of stool during a given period. For example, a subject may have a bowel movement in the morning, and another bowel movement in the night.

It must be noted that, as used in the specification, the singular forms “a,” “an” and “the” include plural referents unless the context clearly dictates otherwise.

The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements).

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used shall only be interpreted as indicating exclusive alternatives (i.e. “one or the other but not both”) when preceded by terms of exclusivity, such as “either,” “one of,” “only one of,” or “exactly one of.”

### II. System Environment Overview

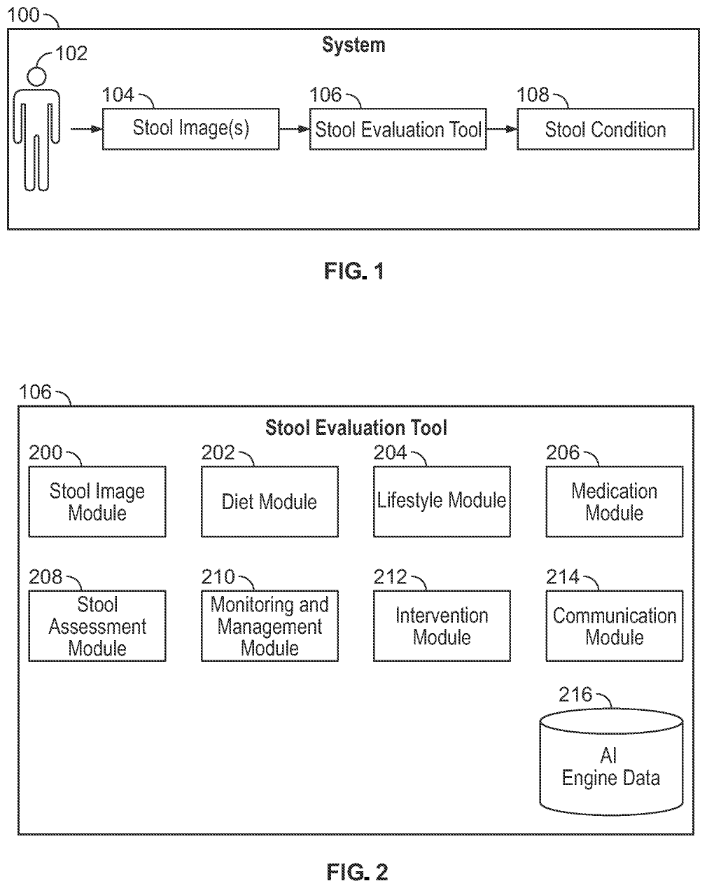

Described herein, in some embodiments, are systems and methods for determining and/or monitoring a stool condition for a subject. FIG. 1 depicts an overview of an exemplary system 100 for determining and/or monitoring a stool condition for a subject 102. In some embodiments, the system 100 receives one or more images of a stool 104 from an image capturing device that is then used by a stool evaluation tool 106 to determine a stool condition 108 for the subject 102. Exemplary image capturing devices include, for example, a standalone camera (configured to be operatively communicated with a computing device), a mobile device (as described herein, such as a smartphone, tablet, smart watch, etc.), a laptop, a desktop, or others known in the art. In some embodiments, the stool is expelled by the subject into a receptacle (e.g., located in a toilet, basin, ground, or other location).

As described herein, in some embodiments, determining the stool condition 108 includes performing a stool assessment to a) characterize the stool, and/or b) identify one or more medical conditions, illnesses and/or diseases based on the image of the stool (stool image). In some embodiments, the stool evaluation tool 106 is configured to determine an efficacy and/or impact on a stool condition 108 based on a) existing diet, b) change in diet, c) existing lifestyle (e.g., exercise, sleep), d) change in lifestyle, e) medications, f) change in medication, and/or any combination thereof. In some embodiments, the stool evaluation tool 106, based on the stool image 104, is configured to determine an intervention to help alleviate any symptoms related to the stool condition 108 experienced by the subject, and/or to help reduce the risk of the subject experiencing any symptoms related to the stool condition 108.

In some embodiments, the stool condition 108 is based on an aggregate of stool assessments performed on stool for one or more bowel movements. For example, the stool condition may be based on stool from a single bowel movement, or from a plurality of bowel movements over a period of time (e.g., over 1, 2, 3, 4, 5, 6, 7, 15, 30, 60, 90, 180, 360, or more days).

In some embodiments, the system 100 provides an integrated management tool for determining and/or monitoring a stool condition 108 for the subject, and for communicating to the subject and/or a healthcare provider (e.g., physician, nurse, or any other medical professional) the stool condition, stool assessments, preferred subject conditions for a stool condition, and/or interventions based on the stool condition. With reference to FIG. 1, as described herein, the stool image(s) 104 for a subject 102 (e.g., obtained via an image capture device) are received by the stool evaluation tool 106, which then determines a corresponding stool condition 108. In some embodiments, the stool condition 108 is output onto a display interface (e.g., a monitor, screen, smart device screen, etc.).

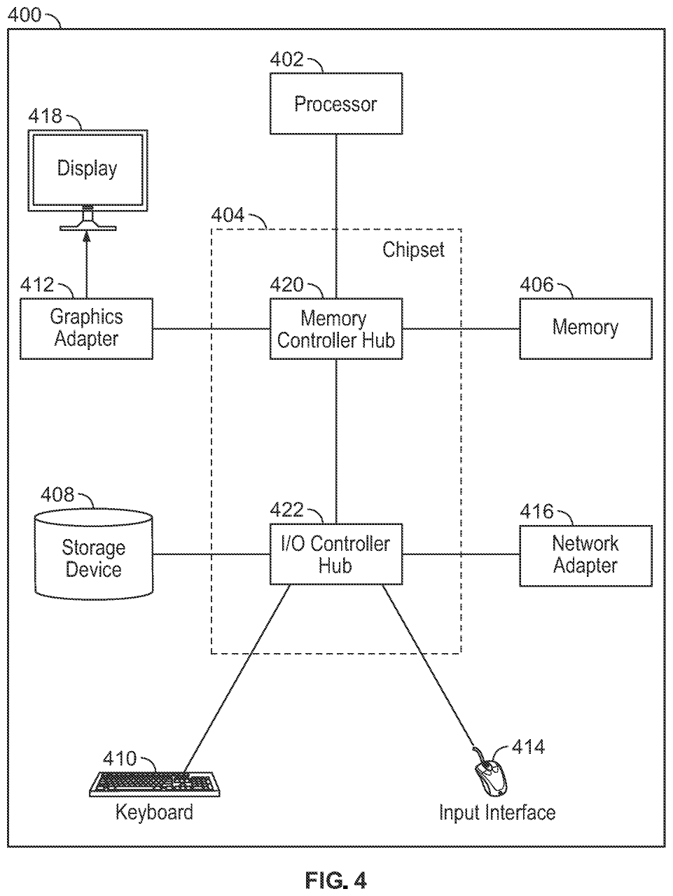

In some embodiments, the stool evaluation tool 106 is provided by one or more computing devices, wherein the stool evaluation tool 106 can be embodied as a computer system (e.g., see FIG. 4, reference character 400). Accordingly, in some embodiments, methods and steps described in reference to the stool evaluation tool 106 are performed in silico. For example, in some embodiments, the stool evaluation tool 106 is configured to apply one or more artificial intelligence (“AI”) engines (e.g., trained models, decision trees, analytical expressions, etc.) so as to determine the stool condition 108. In some embodiments, the one or more AI engines each apply an algorithm, such as a machine learning algorithm (as described herein), to the one or more stool images 104 obtained. In some embodiments, the image capture device and the stool evaluation tool are provided by the same computing device (for e.g., same mobile device, laptop, etc.). As described herein, in some embodiments, the computing device is in operative communication with a remote computing device (including a remote server). In some embodiments, subjective, self-assessments of stool characterization may result in inconsistent and/or inaccurate determinations of a stool condition. Accordingly, in some embodiments, using an AI engine helps increase the accuracy and consistency in determining a stool condition, as described herein.

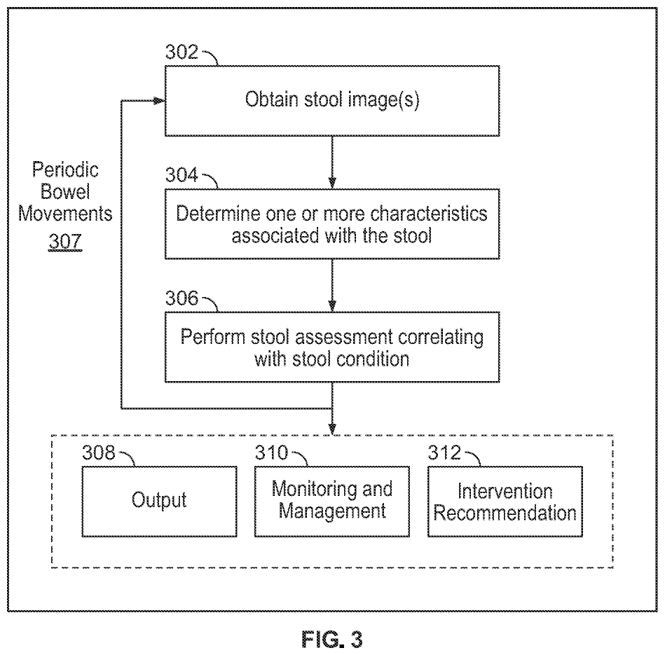

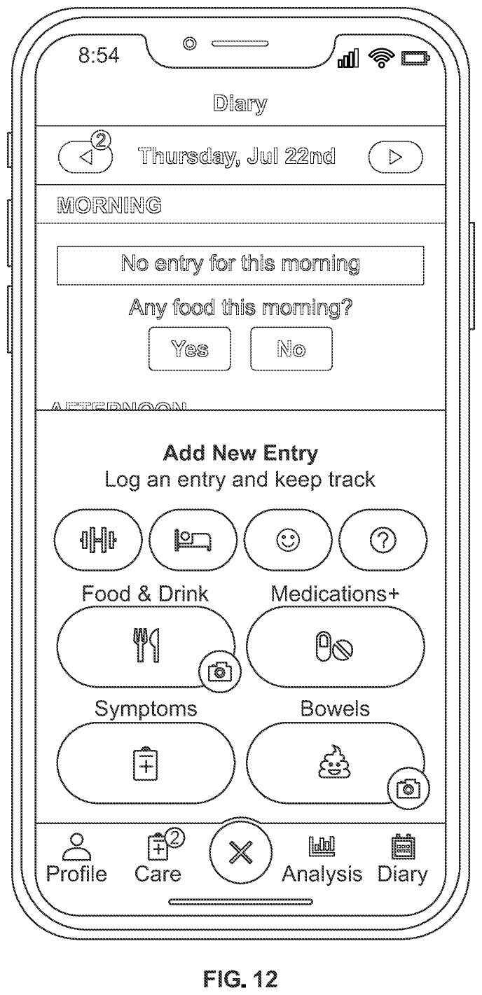

With reference to FIG. 2, a block diagram is depicted illustrating exemplary computer logic components of the stool evaluation tool 106, in accordance with an embodiment. Here, the stool evaluation tool 106 includes a stool image module 200, a diet module 202, a lifestyle module 204, a medication module 206, a stool assessment module 208, a monitoring and management module 210, an intervention module 212, a communication module 214, and an Artificial Intelligence (“AI”) engine data storage 216. In some embodiments, the stool evaluation tool 106 can be configured differently with additional or fewer modules. For example, a stool evaluation tool 106 need not include the intervention module 212. In some embodiments, the AI module 208 and/or the AI engine data storage 218 are located on a different tool and/or computing device. As described herein, in some embodiments, the stool evaluation tool 106 is provided with a computing device, such as a mobile device (e.g., smartwatch, smartphone, tablet, etc.). In some embodiments, the communication module 214 is configured to allow a subject to communicate with the a healthcare administrator (and vice versa). FIG. 12 provides an exemplary depiction of display of a mobile device with the stool evaluation tool 106.

### Stool Condition

As described herein, in some embodiments, systems and methods herein are configured to determine a stool condition 108 for a stool from a subject 102 based on an image 104 of said stool. In some embodiments, the stool condition 108 comprises a) characterizing the stool based on a stool assessment performed by a machine learning algorithm, and/or b) identifying one or more medical conditions, illnesses, and/or diseases based on a stool assessment performed.

In some embodiments, the stool condition 108 is based on a plurality of characteristics associated with the stool in the image(s) 104 of the stool (stool image(s)). In some embodiments, the plurality of characteristics of the stool comprise i) shape and texture, ii) consistency, iii) fragmentation, iv) fuzziness, and/or v) volume. Table 1 below provides a summary of each characteristic.

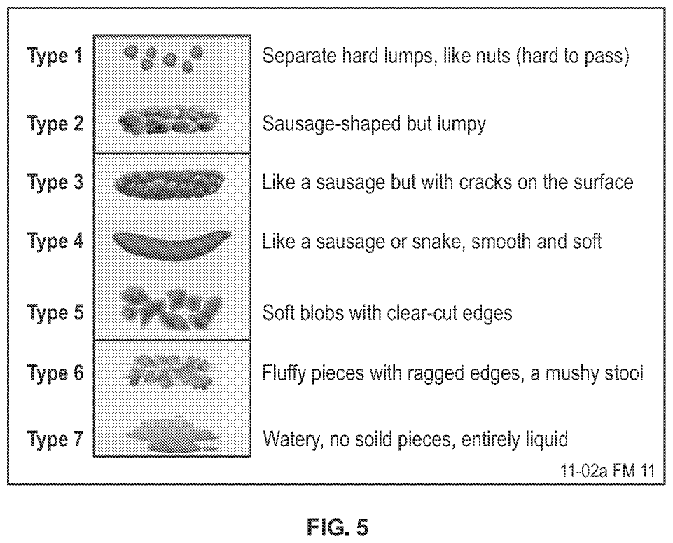

TABLE 1

| Illustrative | |
|---|---|
| variables | Illustrative definition |
| Shape and | Stool can be assigned to various categories based on the |
| Texture | shape of the stool and texture. An exemplary categorical |
| | classification includes the Bristol Stool Scale ( see |
| | FIG. 5 for exemplary categories). |
| Consistency | A liquid-to-solid scale. 0 may correspond to pure liquid, |
| | in which not a single solid piece can be seen. 100 may |
| | correspond to a complete solid. |
| Fragmentation | A scale indicating to what degree the stool is broken up |
| | into different pieces. Fragmentation = 0 may mean there |
| | is only 1 piece of stool. Fragmentation = 100 may mean |
| | there are many pieces of stool. |
| Fuzziness | A scale indicating clarity of boundaries of the stool. A |
| | clear boundary may be a clear straight line between the |
| | stool and the background. Fuzziness = 0 may mean the |
| | lines are clear, Fuzziness = 100 may mean the stool |
| | and the water are indistinguishable |
| Volume | A scale indicating stool size. A small pebble of stool |
| | may be considered 0, a normal size stool may be |
| | considered 50, and a very large stool may be considered |
| | 100. |
| Other suitable | Other suitable definitions |
| variables | |

As described herein, the shape and texture characteristic provides categories according to which the stool is classified as. The shape and texture characteristics may correlate with a bowel movement symptom of the subject, such as diarrhea, constipation, indigestion, intestinal bleeding, incomplete evacuation, etc. In some embodiments, the shape and texture may correlate with having normal digestive health. In some cases the shape refers to the general shape of the stool (e.g., flat, lumpy, sausage type), and how the shape is allocated (e.g., multiple pieces). In some cases, the texture correlates with how hard or soft the stool is, and/or liquid to solid make-up. As described herein, the Bristol Stool Scale may be an exemplary scale for the shape and texture characteristic (see FIG. 5, types 1 to 7).

Accordingly, in some embodiments, the stool assessment module 208 performs a stool assessment that determines the plurality of characteristics of the stool, and determines a corresponding score and/or rating for each of the characteristics.

As described herein, in some embodiments, the stool assessment module 208 uses one or more artificial intelligence (“AI”) engines (e.g., which may include one or machine learning algorithms) to perform the stool assessment. In some embodiments, the AI engine(s) access the AI engine data when performing the stool assessment to determine a score and/or rating. For example, as described herein, the AI engine data may include trained data, such as at least hundreds or thousands of images of stool having a score and/or rating for one or more of the characteristics. In some embodiments, the images of stool were annotated with said score and/or rating. Accordingly, in determining the plurality of characteristics, the AI engine may correlate the stool image(s) 104 with the images from the AI engine data to identify a respective score and/or rating for each characteristic, thereby determining a stool condition. In some embodiments, additional images of stool may be manually classified and provided to the AI engine data. For example, in some embodiments, annotators (e.g., subject, healthcare administrator, or other third party) may classify stool based on visual annotation rules (e.g., a guide) that define each increment of values (e.g., score and/or rating) for the characteristics. In some embodiments, the guide may include 1, 2, 3 or more illustrative images of stool for each incremental value (on the score and/or rating) of each characteristic. Below is an exemplary score and/or rating for the plurality of characteristics, wherein the shape and texture characteristic is provided according to the Bristol Stool Score.
A. Bristol Scale 1-7 values
B. Consistency (0-100 values in increments of 10)
C. Fragmentation (0-100 values in increments of 10)
D. Fuzziness (0-100 values in increments of 10)
E. Volume (0-100 values in increments of 10)

In some embodiments, a preferred category scale for the Bristol Stool Scale is from 3 to 5, such as 4. In some embodiments, a preferred scoring range for consistency is from about 30 to about 70, such as from about 40 to about 60, or 50. In some embodiments, a preferred scoring range for fragmentation is from about 0 to about 30, such as from about 0 to about 20, or 0. In some embodiments, a preferred scoring range for fuzziness is from about 0 to about 30, such as from about 0 to about 20, or 0. In some embodiments, a preferred range for volume depends on each case. In some embodiments, a high volume score, such as from 70-100 is preferred to show good passage of bowel movement. In some embodiments, a moderate volume score, such as from about 40-80 is preferred.

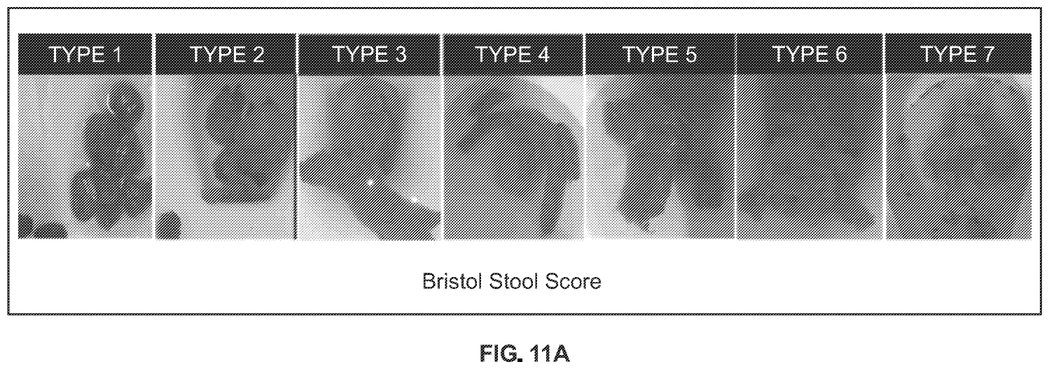

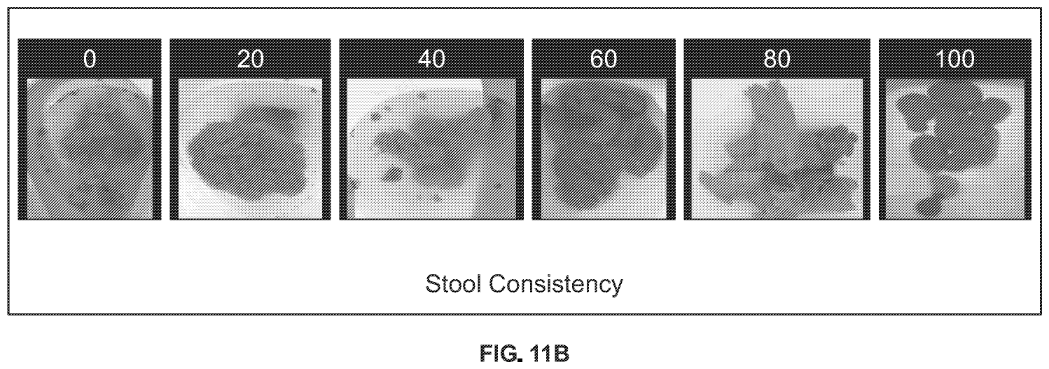

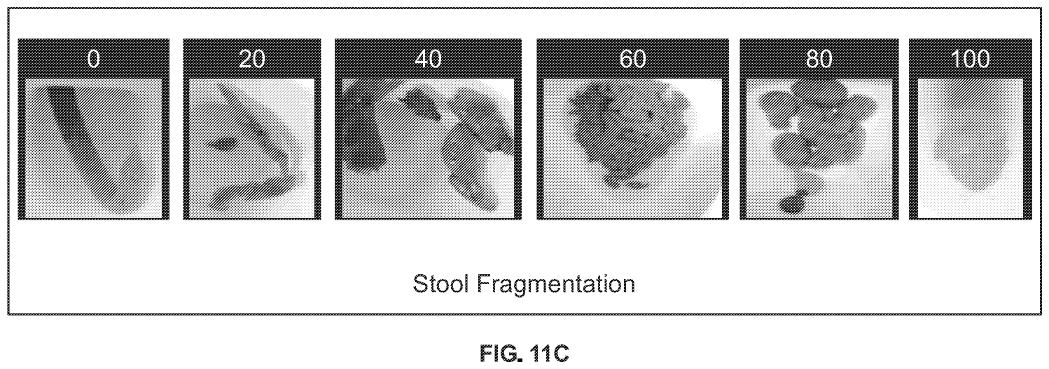

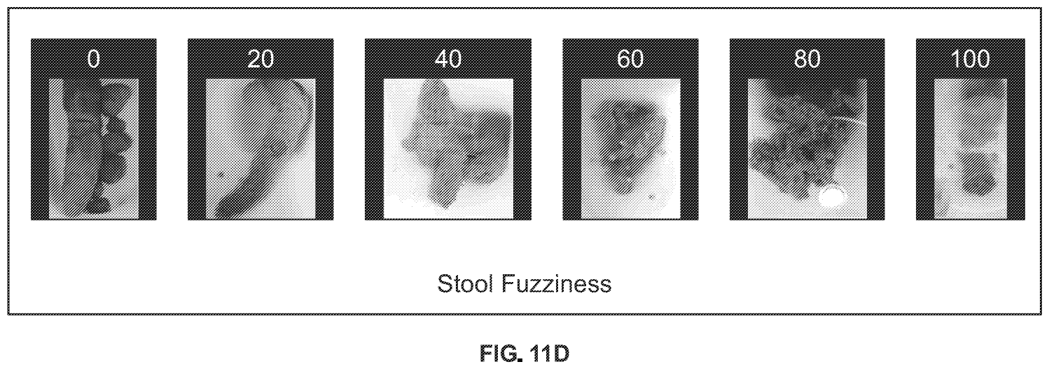

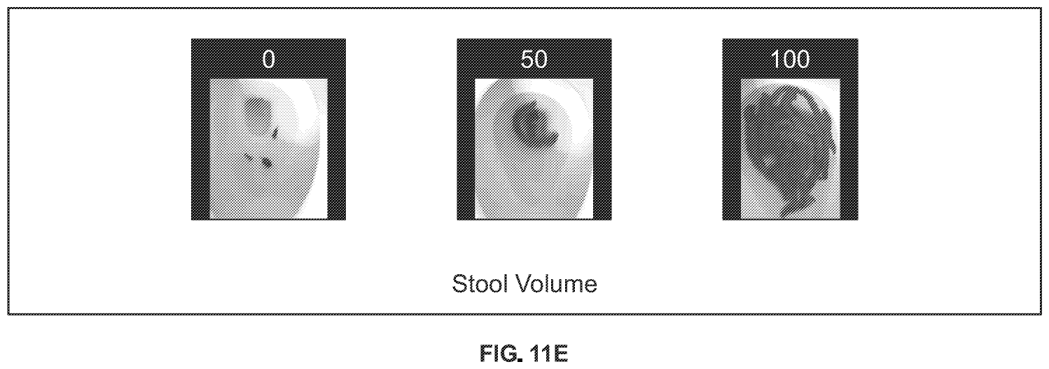

Table 1 described herein provides an exemplary description of the characteristics and what each ends of the scoring scale represents. FIGS. 11A-E further provides exemplary images for the characteristics at different increments.

In some embodiments, the stool assessment module 208 performs a stool assessment that identifies one or more medical conditions, illnesses, and/or diseases based on the stool image(s) 104. In some embodiments, the stool assessment comprises using the plurality of characteristics described herein, and/or one or more stool factors. In some embodiments, the one or more stool factors comprise blood found in the stool, amount of blood found in the stool, color of blood in the stool, degree to which blood is embedded within the stool and/or is outside the stool in the toilet bowl, color of the stool, amount of mucus on the stool, diameter of the stool, buoyancy of the stool, or any combination thereof. In some embodiments, identifying the one or more medical conditions, illnesses, and/or diseases is based on several bowel movements over a period of time (e.g., over a number of days, weeks, months, etc.). In some embodiments, the AI engine (as part of the stool assessment module 208) accesses the AI engine data (e.g., trained data) to correlate the plurality of characteristics and/or one or more stool factors to identify the one or more medical conditions, illnesses, and/or diseases. In some embodiments, the one or more medical conditions, illnesses, and/or diseases comprise ulcerative colitis, hepatic encephalopathy, irritable bowel syndrome, Crohn's disease, or any combination thereof. For example, for stools having blood found therewith, the AI engine (using a machine learning algorithm for example, as described herein), may correlate the blood and optionally one or more of the stool characteristics (as described herein) to ulcerative colitis. In some embodiments, one or more stool factors are able to correlate with a physiological event. For example, in some embodiments, a color of blood found with the stool may correlate with a location along the gastrointestinal tract where bleeding is occurring.

### Stool Image Module

In some embodiments, the stool is expelled by the subject into a receptacle. In some embodiments, the receptacle comprises a toilet, a basin, the ground and/or any other suitable receptacle. In some embodiments, the stool includes stool expelled during a bowel movement session. In some embodiments, the stool includes stool expelled during multiple bowel movement sessions. For example, a first bowel movement session may be during the morning, and a second bowel movement session may be at night.

As described herein, in some embodiments, one or more images 104 of the stool is captured using an image capture device. In some embodiments, the image capture device comprises a camera. In some embodiments, the camera is part of a computing device, such as for example a mobile device, a desktop, a laptop, etc. In some embodiments, the mobile device comprises a smartphone, a smartwatch, a tablet, etc. In some embodiments, the camera is in operative communication and/or configured to be in operative communication with a computing device (e.g., via a wired and/or wireless connection). In some embodiments, the camera is configured to transfer the stool image(s) 104 to a computing device, e.g., using a memory storage stick or device, or other devices as known in the art.

In some embodiments, the image(s) are captured by a first party (for example, the subject 102, a medical professional, or any other person). In some embodiments, the image capture device is part of another computing device, and communicated to the stool evaluation tool 106. For example, a first party (for example, the subject 102, a medical professional, or any other person) uses an image capture device (as described herein) to capture one or more images of a stool, wherein the image(s) are then provided to a second party (for example, the subject 102, a medical professional, or any other person different from the individual operating the image capture device), which implements the stool evaluation tool 106 to determine the stool condition 108.

In some embodiments, the image capture device (also interchangeably referred to image acquisition device) is in operative communication with the stool image module 200. In some embodiments the stool image module 200 receives the image(s) 104 obtained via the image capture device.

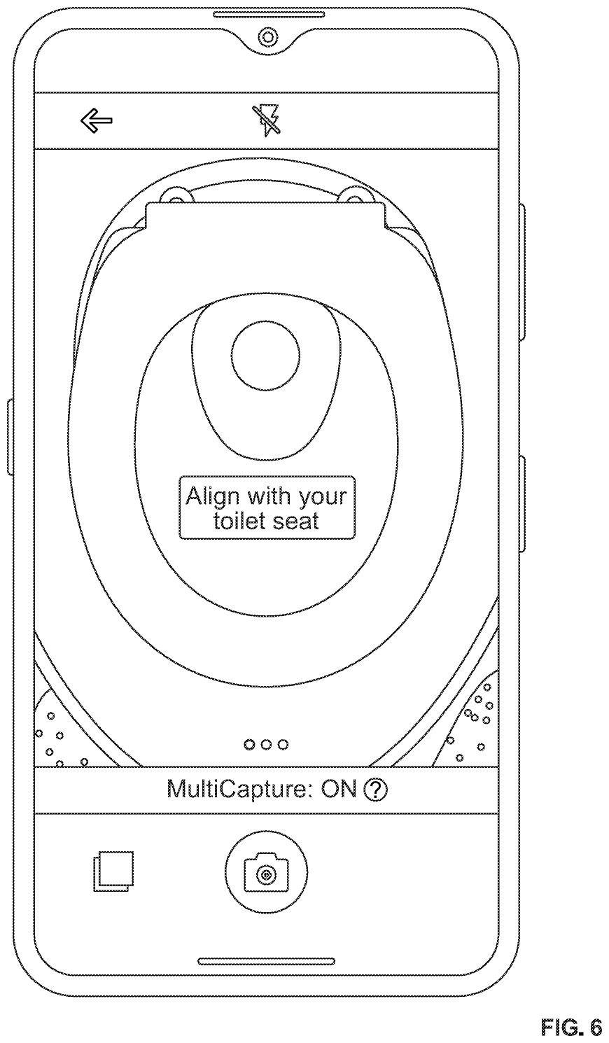

In some embodiments, the image capture device includes a display and/or is in operative communication with a display. In some embodiments, the display provides one or more guiding features to allow an acceptable image of the stool to be captured. For example in some embodiments, the image of the stool must be entirely captured to be acceptable. In some embodiments, the guiding features allow for an equidistant image to be captured. In some embodiments, the guiding feature is configured to align with the receptacle. For example, in some embodiments, the guiding features includes a toilet seat depicted on the display that is configured to align with an actual toilet seat of a toilet acting as the receptacle (see FIG. 6 for example). In some embodiments, the guiding feature includes a depiction of a toilet seat having a transparent center portion to capture stool located within the actual toilet.

In some embodiments, the stool image module 200 includes an image recognition module configured to detect whether an image of stool has been captured or not. For example, if the captured image does not include any stool portion (or a minimal amount of stool), the stool image module 200 may indicate (e.g., via a display) that an image of a stool was not captured.

In some embodiments, the stool image module 200 includes a cropping tool. The cropping tool may automatically crop out of image elements that are not stool. The stool image module may include a zoom function, a brightness function, a contrast function, a digital filtering function, and/or any other suitable functions. In some embodiments, one or more of the functions may provide a view of stool that compensates for different ambient lighting.

In some embodiments, the one or more images of the stool capture are received and/or stored by the stool image module 200. In some embodiments, the images are stored in a location on the computing device that is not a camera roll. In some embodiments, the images are hidden behind a security feature for privacy.

In some embodiments, the one or more images of the stool are associated with a date and time received by the stool image module 200. In some embodiments, multiple images of the same stool are obtained. In some embodiments, the images are acquired by execution of a “click.” The click may be a digital shutter click. In some embodiments, the stool image module acquires 1, 2, 3 or any suitable number of images of the stool per click. The images associated with a click may be acquired from different angles relative to the stool. The images corresponding to the click may be used to increase the diversity of data available for training AI models. The images corresponding to the click, may be used to increase the diversity of data available for training AI models without requiring the user to photograph additional stool.

In some embodiments, the stool image module 200 is in operative communication with a user interface so as to receive input from the subject and/or healthcare administrator. In some embodiments, the user interface allows the subject and/or healthcare administrator provide metadata about the stool. In some embodiments, the user interface allows for the subject and/or healthcare administrator to annotate the image. A quality assurance (“QA”) process may involve multiple annotators. The user interface may also allow the subject and/or healthcare administrator to conveniently sort through the image history and data.

### Subject Conditions

In some embodiments, the stool evaluation tool 106 comprises one or more subject conditions used by the stool evaluation tool 106 for performing a stool assessment, including monitoring or managing the stool condition of a subject over a period of time. In some embodiments, the one or more subject conditions comprises diet conditions, lifestyle conditions, and/or medication conditions. In some embodiments, the diet conditions are received by the stool evaluation tool 106 via the diet module 202. In some embodiments, the diet conditions comprise food and/or liquid intake by the subject 102. For example, in some embodiments, the diet conditions comprise the types of food and/or liquid ingested by the subject 102. In some embodiments, the diet conditions are inputted by the subject and/or another party (health administrator, other family member of the subject, etc.). In some embodiments, the diet conditions are inputted via a user interface. In some embodiments, the diet conditions are obtained via the image capture device, using an image recognition module. For example, in some embodiments, the image recognition module is configured to detect the type of food from the image of said food.

In some embodiments, the diet module 202 is configured to extract one or more characteristics of each type of food and/or liquid ingested. For example, in some embodiments, the diet module 202 is configured to detect one or more ingredients of the food and/or liquid, such as containing rice, meat, dairy, beans, etc.

In some embodiments, each diet condition inputted and/or received is stored on the diet module 202. In some embodiments, each diet condition inputted and/or received is associated with a date and time of ingestion by the subject 102.

In some embodiments, the lifestyle conditions are received by the stool evaluation tool via the lifestyle module 204. In some embodiments, the lifestyle conditions comprise activity by the subject, such as amount of sleep, amount of exercise, stress, etc., experienced by the subject. In some embodiments, the lifestyle conditions are inputted by the subject and/or another party (health administrator, other family member of the subject, etc.). In some embodiments, the lifestyle conditions are inputted via a user interface. In some embodiments, the lifestyle conditions are obtained via another smart device (e.g., a smartwatch, smartphone, exercise device (e.g., FITBIT®).

In some embodiments, each lifestyle condition inputted and/or received is stored on the lifestyle module 204. In some embodiments, each lifestyle condition inputted and/or received is associated with a date and time of occurrence by the subject.

In some embodiments, the medication conditions are received by the stool evaluation tool via the medications module 206. In some embodiments, the medication conditions comprise medications intake by the subject 102. For example, in some embodiments, the medication conditions comprises the types of medications ingested by and/or otherwise administered to the subject 102. In some embodiments, the medication conditions are inputted by the subject and/or another party (health administrator, other family member of the subject, etc.). In some embodiments, the medication conditions are inputted via a user interface.

In some embodiments, each medication condition inputted and/or received is stored on the medication module 206. In some embodiments, each medication condition inputted and/or received is associated with a date and time of ingestion by and/or administration to the subject.

### Stool Assessment Module

As described herein, in some embodiments, the stool assessment module 208 is configured to determine a stool condition 108 for a stool of a subject 102, based on one or more images 104 of the stool. In some embodiments, stool assessment module 208 performs a stool assessment to characterize the stool, and/or to identify one or more medical conditions, illnesses, and/or diseases associated with the stool. In some embodiments, as described herein, the stool assessment module 208 is configured to determine one or more characteristics of the stool, and assigns a scores and/or rating to the characteristics (via a stool assessment). In some embodiments, the stool assessment module 208 uses one or more artificial intelligence engines to assign the score and/or rating. In some embodiments, the AI engine (which may use one or more machine learning algorithms), accesses the AI engine data so as to perform the stool assessment. Similarly, in some embodiments, the stool assessment module 208 identifies one or more medical conditions, illnesses, and/or diseases associated with the stool based on the plurality of characteristics of the stool (as described herein), and/or one or more stool factors, such as presence of blood with the stool, color of the stool, etc. In some embodiments, the plurality of characteristics and/or one or more stool factors are communicated to a health administrator (e.g., via the communication module 214) for diagnosing and/or identifying a medical condition (e.g., irritable bowel syndrome, Crohn's disease, etc.). In some embodiments, the healthcare administrator (e.g., medical professional, physician, nurse, gastroenterologist, and/or dietitian) is able to review the stool assessment(s) and provide a recommendation for a treatment or other care.

In some embodiments, the determined stool condition 108 is a point-in-time analysis of the stool. In some embodiments, stool conditions from several bowel movement sessions over a period of time (e.g., days, weeks, months, etc.) provides an overall synopsis of a health condition by the subject based on stool. In some embodiments, the Monitoring and Management Module 210 helps monitor the stool condition of a subject over time. In some embodiments, each stool assessment performed is stored in the MM module, and optionally associated with a corresponding date and time relating to the bowel movement.

In some embodiments, the stool assessment module 208 incorporates a validation step to help increase the accuracy of a stool condition determination (via a stool assessment). In some embodiments, the validation step comprises the stool assessment module 208 performing multiple separate stool assessments on the same stool image (e.g., via the AI engine), so as to compare the scores and/or ratings assigned for the stool, and/or the one or more stool factors identified. In some embodiments, the stool assessment module 208 performs at least 2, 3, 4, 5, 6, 10, or 20 stool assessments on a given stool image. For example, in some embodiments, each stool assessment may be performed using a different machine learning algorithm (as described herein). Accordingly, in some embodiments, if the score and/or rating any given characteristic is within a minimum tolerance for a number of the stool assessments, the determined stool condition has been validated. In some embodiments, if the score and/or rating of any given characteristic is outside a minimum tolerance for one or more of the stool assessments, the determined stool condition fails validation. In some embodiments, the minimum tolerance is based on a standard deviation, a mean, median, or any combination thereof of the values (e.g., score) of each characteristic for the plurality of stool assessments. In some embodiments, such validation includes an ensemble prediction method to generate a confidence score for each stool assessment performed, where multiple trained iterations of a neural network are run on each image (e.g., multiple stool assessments are performed) and the variance amongst the resulting stool assessments is an indicator of predictive confidence.

In some embodiments, the validation step is based on two or more different images of the same stool, wherein if the scale and/or rating any given characteristic is with a minimum tolerance for a number of the stool assessments (of the different stool images), the determined stool condition has been validated. In some embodiments, if the scale and/or rating of any given characteristic outside a minimum tolerance for one or more of the stool images, the determined stool condition has failed validation.

In some embodiments, wherein a characteristic of the stool has failed validation, the stool assessment module 208 is configured to communicate to the subject or healthcare administrator (e.g., physician, nurse, etc.) of the stool condition failing validation. For example, in some embodiments, the stool assessment module 208 communicates to the healthcare administrator (e.g., gastroenterologist) via the communication module 214, as described herein. In some embodiments, the plurality of characteristics, and/or the specific characteristic(s) for which validation is failing is flagged to the subject and/or healthcare administrator. In some embodiments, the subject and/or healthcare administrator is able to view the stool image(s), and provide said image with a score and/or rating for the characteristic(s). In some embodiments, the stool assessment module 208 is then configured to receive the manually inputted score and/or rating, and determine the stool condition accordingly.

In some embodiments, when validation of a stool assessment has passed, wherein each stool assessment includes i) a stool assessment for each of multiple images of a stool, or ii) multiple stool assessments using different machine learning algorithms, the stool evaluation tool will output a single stool assessment. In some embodiments, the stool assessment (e.g., score for each characteristic) is based on an average score for each characteristic, a median score, the best score, the worst score, any statistical evaluation known in the art, or any combination thereof.

In some embodiments, the stool assessment module is configured to automatically perform a stool assessment upon receiving one or more images of a stool.

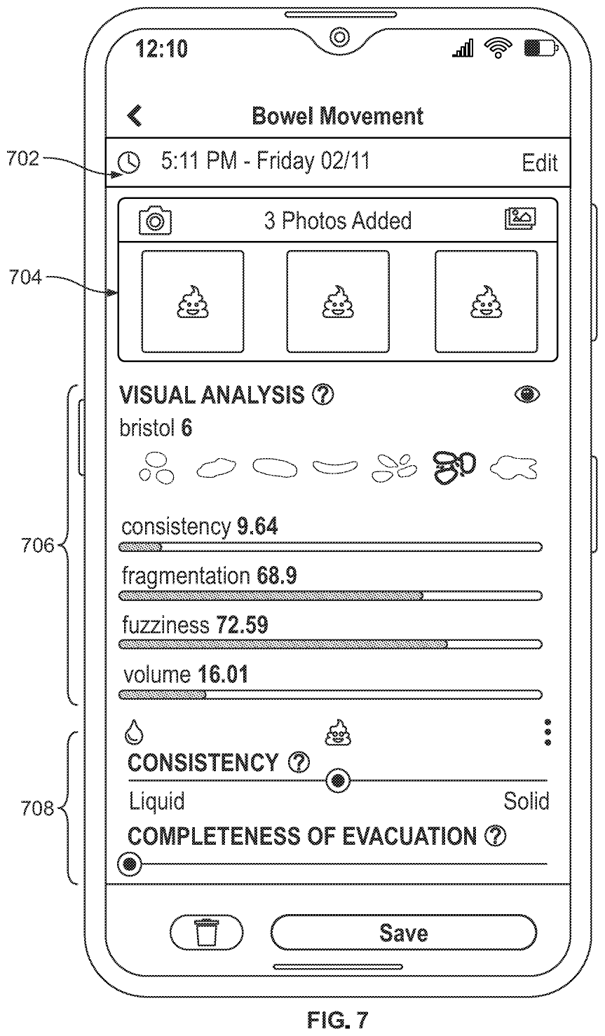

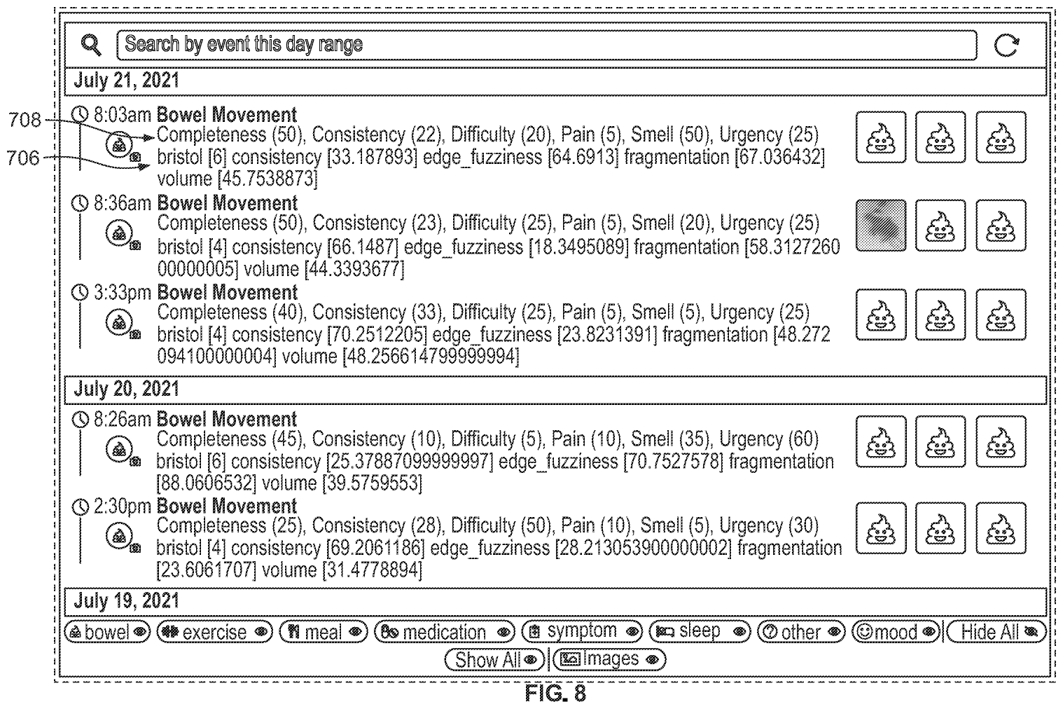

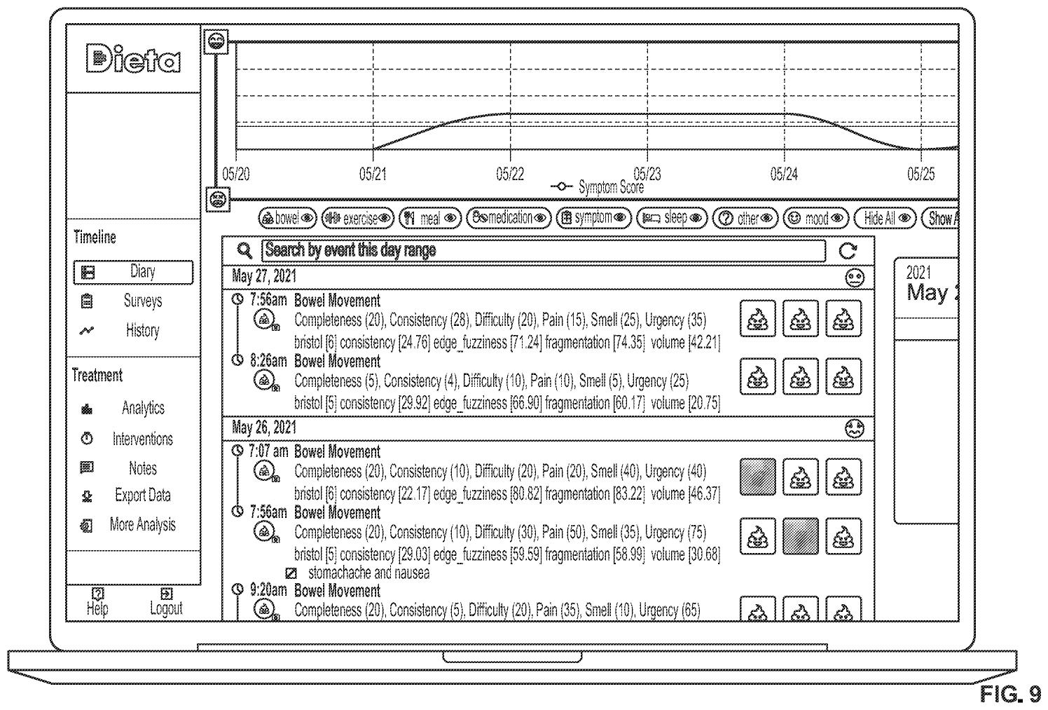

In some embodiments, the stool assessment module 208 is in communication with a display of a computing device (as described herein), or a different computing device that may be located remote (e.g., by a healthcare administrator). In some embodiments, the stool assessment module is configured to output the determined stool condition 108 to said display. FIG. 7 provides an exemplary stool assessment (and thereby stool condition) depiction outputted onto a display for a given bowel movement. As depicted, the output includes the date and time of when the bowel movement occurred 702, one or more photos relating to the stool 704, as well as an exemplary stool assessment 706 comprising the plurality of characteristics such as shape and texture (in this example, the shape and texture characteristic was identified with the Bristol Stool Scale), consistency, fragmentation, fuzziness, and volume. In some cases, output further provides an interface for a subject to perform a self-assessment relating to certain stool characteristics and/or bowel movements. For example, in some embodiments, the self-assessment properties 708 include a self-assessed consistency, completeness of the evacuation, difficulty to pass (FIG. 8), pain of passing the stool (FIG. 8), smell of the stool (FIG. 8), and/or urgency of the bowel movement (FIG. 8).

FIG. 8 provides an exemplary output of stool condition determined for multiple bowel movements, wherein each stool associated with a bowel movement is listed according to date and time. As depicted, the stool assessment performed 706, comprising the plurality of characteristics, is provided with each listed bowel movement. In some embodiments, additional self-assessed properties 708 relating to the stool and/or bowel movements are provided. In some embodiments, additional features relating to the bowel movement are provided, such as i) difficulty to pass the stool, ii) pain in passing the stool, iii) smell of the stool, and iv) urgency in expelling the stool, all of which may be a part of the stool assessment. In some embodiments, one or more images of the stool for the corresponding bowel movement is also depicted. FIG. 9 provides another exemplary output of stool condition, which depicts the output shown in FIG. 8, along with other features, such as a graph depicting a trend in the symptoms over time, and sidebar tools to access other modules in the stool evaluation tool.

### Artificial Intelligence (“AI”) Engine(s) and AI Engine Data

As described herein, in some embodiments, the stool evaluation tool 106 is configured to determine a stool condition 108 for a subject 102. In some embodiments, the stool evaluation tool 106, via the stool assessment module 208, applies one or more images 104 of stool obtained for the subject to one or more AI engines to determine the stool condition 108.

In some embodiments, the AI engine includes one or more algorithms to determine a stool condition 108 based on the image(s) 104 of stool received (as described herein). In some embodiments, each algorithm may correspond to identifying one or more characteristics (as described herein) of the stool. As described herein, in some embodiments, the one or more characteristics is used by the stool assessment module 208 so as to determine the stool condition 108, such as for example, determining a characterization of the stool and/or identifying an illness, medical condition, and/or disease correlating with the stool image 104. In some embodiments, the one or more AI engines apply algorithms (e.g., algorithms embodied in trained models) to correlate the image(s) of the stool with the various characteristics (as described herein) using trained data found in the AI engine data 216. In some embodiments, at least one of the one or more algorithms may comprise a machine learning algorithm incorporating artificial intelligence (AI) to help improve accuracy of said stool condition determination. For example, in some embodiments, said AI is applied to the trained model data (e.g., which may be in the AI engine data 216) and optionally past images of stool specifically from the subject and that were vetted (e.g., by a physician or other medical professional) to identify the characteristics of the stool.

In some embodiments, any one of the AI engine(s) described herein is any one of a regression model (e.g., linear regression, logistic regression, or polynomial regression), decision tree, random forest, gradient boosted machine learning model, support vector machine, Naïve Bayes model, k-means cluster, or neural network (e.g., feed-forward networks, convolutional neural networks (CNN), deep neural networks (DNN), autoencoder neural networks, generative adversarial networks, or recurrent networks (e.g., long short-term memory networks (LSTM), bi-directional recurrent networks, deep bi-directional recurrent networks), or any combination thereof. In particular embodiments, any one of the AI engine(s) described herein is a logistic regression model. In particular embodiments, any one of the AI engine(s) described herein is a random forest classifier. In particular embodiments, any one of the AI engine(s) described herein is a gradient boosting model.

In some embodiments, any one of the AI engine(s) described herein (e.g., a trained model) can be trained using a machine learning implemented method, such as any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, and dimensionality reduction techniques such as manifold learning, principal component analysis, factor analysis, autoencoder regularization, and independent component analysis, or combinations thereof. In particular embodiments, the machine learning implemented method is a logistic regression algorithm. In particular embodiments, the machine learning implemented method is a random forest algorithm. In particular embodiments, the machine learning implemented method is a gradient boosting algorithm, such as XGboost. In some embodiments, any one of the trained model(s) described herein is trained using supervised learning algorithms, unsupervised learning algorithms, semi-supervised learning algorithms (e.g., partial supervision), weak supervision, transfer, multi-task learning, or any combination thereof.

In some embodiments, any one of the trained model(s) described herein has one or more parameters, such as hyperparameters or model parameters. Hyperparameters are generally established prior to training. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters are generally adjusted during training. Examples of model parameters include weights associated with nodes in layers of neural network, support vectors in a support vector machine, node values in a decision tree, and coefficients in a regression model. The model parameters of the risk prediction model are trained (e.g., adjusted) using the training data to improve the predictive capacity of the risk prediction model.

In some embodiments, any one of the trained model(s) described herein are trained via training data located in the trained model data (which may be included with the decision engine module 218).

In various embodiments, the training data used for training any one of the trained model(s) described herein includes reference ground truths that indicate that a training stool image was identified with a particular characteristic and/or a strong showing of a particular characteristic (hereafter also referred to as “positive” or “+”) or whether the training stool image was not identified with a particular characteristic and/or was identified with a low prominence of a particular characteristic (hereafter also referred to as “negative” or “−”). In various embodiments, the reference ground truths in the training data are binary values, such as “1” or “0.” For example, a training individual where the stool image was correlated with a medical condition can be identified in the training data with a value of “1” whereas a training individual where the stool image was not correlated with a medica condition can be identified in the training data with a value of “0.” In various embodiments, any one of the trained model(s) described herein are trained using the training data to minimize a loss function such that any one of the trained model(s) described herein can better predict the outcome based on the input (e.g., extracted features of the subject's health parameters). In some embodiments, the loss function is constructed for any of a least absolute shrinkage and selection operator (LASSO) regression, Ridge regression, or ElasticNet regression. In some embodiments, any one of the trained model(s) described herein is a random forest model, and is trained to minimize one of Gini impurity or Entropy metrics for feature splitting, thereby enabling any one of the trained model(s) described herein to more accurately determine a stool condition in the subject.

In various embodiments, the training data can be obtained and/or derived from a publicly available database. In some embodiments, the training data can be obtained and collected independent of publicly available databases. Such training data can be a custom dataset.

In some embodiments, AI engine data storage includes images of stool that have been characterized (for e.g., based on the plurality of characteristics), and/or correlated with a medical condition. In some embodiments, the AI engine data storage comprises at least 20,000, 50,000, 70,000, 100,000, or 1,000,000 images of stool that have been characterized and/or correlated with a medical condition (as described herein). In some embodiments, the AI engine data storage 216 is updated via communication with an external database, and/or is updated based on images of stool as received from the subject.

In some embodiments, the trained images includes multiple images (e.g., 3 images) of the same stool. In some embodiments, each image may have slight variations from each other, such as due to camera movement, lighting, etc. Accordingly, in some embodiments, a single manual stool assessment applied to an image will be allocated to all the images of the stool, thereby providing more trained data with less manual allocation.

### Monitoring and Management Module

In some embodiments, the stool evaluation tool comprises a monitoring and management (“MM”) module 210 for evaluating an overall condition of a subject based on one or more stool conditions. For example, in some embodiments, the MM module 210 is configured to monitor and trend the stool conditions for one or more bowel movement sessions over a period of time (e.g., at least 1, 2, 3, 4, 5, 7, 10, 15, 30, 60, 90, 180, 360, or 1000 days). In some embodiments, by monitoring stool conditions over a period of time, the MM module provides a general trend and status of a health condition for a subject. For example, in some embodiments, the general trend of the stool condition of a subject over a period of time helps identify and/or confirm one or medical conditions, illnesses, and/or diseases of a subject. In some embodiments, the general trend of the stool condition is communicated to a health administrator for diagnosing and/or identifying a medical condition (e.g., irritable bowel syndrome, Crohn's disease, etc.). In some embodiments, the MM module is configured to identify one or more changes with a stool condition based on sequential stool assessments performed on stool images from corresponding bowel movements.

In some embodiments, the MM module 210 is configured to correlate one or more subject conditions with improved or positive stool conditions. In some embodiments, the MM module 210 accesses the diet module 202, lifestyle module 204, and/or medication module 206 to obtain one or ore subject conditions inputted to the stool evaluation tool 106, and correlate with a corresponding bowel movement session according to the similar date and time period. In some embodiments, the MM module 210 is configured to identify the impact to the stool conditions based on changes to the one or more subject conditions. For example, in some embodiments, the MM module 210 may note that improved sleep and/or lower stress improved the stool conditions (e.g., based on the score and/or rating for the plurality of characteristics). In some embodiments, the MM module 210 is configured to identify particular aspects of one or ore subject conditions that correlate with an improved or regressing stool condition 108. For example, in some cases, a dairy diet may worsen the stool condition 108. Accordingly, in some cases, the MM module 210 will correlate an improving stool condition with a reduction in gluten intake by the subject 102. In some embodiments, the MM module 210 outputs a trend in the stool condition (which may focus on specific characteristics of the stool individually) and compared with specific subject conditions. In some embodiments, the MM module 210 can output a trend in change in the stool condition over time, such as over one or more days, such as at least 2, 3, 5, 7, 15, 20, 30, 60, 90, 180, or 360 days. In some embodiments, the MM module 210 is configured to determine an effectiveness of change in a subject condition with respect to improving a stool condition 108.

In some embodiments, the MM module 210 is configured to determine an effectiveness of a medication in improving a stool condition 108, and/or alleviating symptoms from a medical condition, illness, and/or disease. In some embodiments, the MM module 210 tracks the stool condition of a subject for a number of bowel movements over a period of time prior to the subject taking the medication, while taking the medication, and/or after taking the medication. In some embodiments, the MM module 210 is configured to output a change in one or more characteristics of the stool, and correlate changes resulting from the medication intake. In some embodiments the period of time is +/−3 days, 5 days, 1 week, 2 weeks, 4 weeks, or more before and/or after intake of the medication.

In some embodiments, the MM module 210 is configured to output the monitoring (e.g., trends) of the stool condition (e.g., over a number of bowel movements) to a display (as described herein). In some embodiments, the MM module 210 is configured to communicate to a healthcare administrator (e.g., via the communication module) trends of the stool condition, and any particular correlations with a change in subject condition (including effectiveness of a medication). In some embodiments, the MM module 210 is configured to communicate to a healthcare administrator any flagged alerts, such as a deteriorating stool condition, and/or the identification of a medical condition, illness, and/or disease

### Intervention Module

In some embodiments, the stool evaluation tool comprises an intervention module 212 configured to determine an intervention to help improve a stool condition 108. For example, in some embodiments, the intervention module 212 recommends a change in a subject condition, such as diet and/or lifestyle. In some embodiments, the intervention module 212 recommends a medication or other treatment plan to help improve a stool condition. In some embodiments, the intervention module 212 communicates to a healthcare administrator (e.g., via the communication module) any such recommendations, wherein the healthcare administrator may be required to approve such recommendation.

### III. Methods for Determining a Stool Condition

Embodiments described herein include methods for determining a stool condition for a subject by applying one or more artificial intelligence engines to one or more images of stool. Such methods can be performed by the stool evaluation tool described in FIG. 2. FIG. 3 depicts an example flow diagram 300 for determining a stool condition, in accordance with an embodiment. In some embodiments, the stool image module 200 first obtains 302 one or more images of a stool expelled by a subject during a bowel movement session. In some embodiments, the one or more images are obtained using an image capture device, as described herein. In some embodiments, the stool evaluation tool 106 then determines one or more characteristics 304 associated with stool. For example, in some embodiments, the stool evaluation tool will determine a shape and texture of the stool, a consistency of the stool, a fuzziness of the stool (e.g., distinction of edge of the stool compared to a background), a fragmentation of the stool, and/or a volume of the stool (e.g., how much of the stool). In some embodiments, such determination of the characterization comprises a generalization of each characteristic. For e.g., in determining the fragmentation, the stool evaluation tool 106 determines whether the stool is in a single piece, two pieces, four pieces, or more. In some cases, the stool evaluation tool also identifies one or more stool factors, such as presence of blood in the stool, the color of the stool etc.

The stool evaluation tool 106 then performs a stool assessment 306 that correlates with the stool condition. As described herein, in some embodiments, the stool assessment comprises correlating each of the characteristics with a score and/or rating. In some embodiments, the score and/or rating is correlated by using an artificial intelligence engine, which accesses a trained data set from an AI engine data module 216. In some embodiments, the stool assessment alternatively and/or additionally comprises correlating the plurality of characteristics and one or more stool factors with a medical condition, illness, and/or disease (e.g., via the AI engine).

In some embodiments, the stool condition is based on stool assessments performed for stools obtained from one or more bowel movements 307. In some embodiments, the stool condition is based on the aggregate of the stool assessments performed. Accordingly, the stool condition may continue to adjust with each bowel movement. In some cases, an identification of a medical condition, illness, and/or disease is based on minimum number of bowel movements having stool exhibiting one or more characteristics and/or one or more stool factors (as described herein).

In some embodiments, once the stool condition is determined, the stool evaluation tool 106 then outputs 308 the stool condition (e.g., onto a display). In some embodiments, the stool evaluation tool 106 is configured to monitor 310 a stool condition over time and/or to identify changes to the stool condition. In some embodiments, such monitoring allows for the stool evaluation tool to correlate any changes to the subject conditions (as described herein) to a change in stool condition, and/or determine an effectiveness of a medication with respect to improving a stool condition. In some embodiments, the stool evaluation tool is also configured to provide an intervention recommendation 312 based on a determined stool condition, to help alleviate any symptoms related thereto.

### IV. Computer Implementation

The methods described herein, including the methods of implementing one or more decision engines for determining a stool condition, are, in some embodiments, performed on one or more computers.

For example, the building and deployment of any method described herein can be implemented in hardware or software, or a combination of both. In one embodiment, a machine-readable storage medium is provided, the medium comprising a data storage material encoded with machine readable data which, when using a machine programmed with instructions for using said data, is capable of executing any one of the methods described herein and/or displaying any of the datasets or results (e.g., stool condition) described herein. Some embodiments can be implemented in computer programs executing on programmable computers, comprising a processor and a data storage system (including volatile and non-volatile memory and/or storage elements), and optionally including a graphics adapter, a pointing device, a network adapter, at least one input device, and/or at least one output device. A display may be coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high-level procedural or object-oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of an embodiment. The databases of some embodiments can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

In some embodiments, the methods described herein, including the methods for determining a stool condition, are performed on one or more computers in a distributed computing system environment (e.g., in a cloud computing environment). In this description, “cloud computing” is defined as a model for enabling on-demand network access to a shared set of configurable computing resources. Cloud computing can be employed to offer on-demand access to the shared set of configurable computing resources. The shared set of configurable computing resources can be rapidly provisioned via virtualization and released with low management effort or service provider interaction, and then scaled accordingly. A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 4 illustrates an example computer for implementing the entities shown in FIGS. 1-2, and 10. The computer 400 includes at least one processor 402 coupled to a chipset 404. The chipset 404 includes a memory controller hub 420 and an input/output (I/O) controller hub 422. A memory 406 and a graphics adapter 412 are coupled to the memory controller hub 420, and a display 418 is coupled to the graphics adapter 412. A storage device 408, an input device 414, and network adapter 416 are coupled to the I/O controller hub 422. Other embodiments of the computer 400 have different architectures.

The storage device 408 is a non-transitory computer-readable storage medium such as a hard drive, compact disk read-only memory (CD-ROM), DVD, or a solid-state memory device. The memory 406 holds instructions and data used by the processor 402. The input interface 414 is a touch-screen interface, a mouse, track ball, or other type of pointing device, a keyboard, or some combination thereof, and is used to input data into the computer 400. In some embodiments, the computer 400 may be configured to receive input (e.g., commands) from the input interface 414 via gestures from the user. The network adapter 416 couples the computer 400 to one or more computer networks.

The graphics adapter 412 displays images and other information on the display 418. In various embodiments, the display 418 is configured such that the user may (e.g., subject, healthcare professional, non-healthcare professional) may input user selections on the display 418 to, for example, initiate the system for determining a stool condition. In one embodiment, the display 418 may include a touch interface. In various embodiments, the display 418 can show a stool condition, trends in the stool condition, etc. for the subject and associated monitoring. Thus, a user who accesses the display 418 can inform the subject of the stool condition. In various embodiments, the display 418 can show information such as depicted in FIGS. 6-9.

The computer 400 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 408, loaded into the memory 406, and executed by the processor 402.

The types of computers 400 used by the entities of FIGS. 1-2 and 10 can vary depending upon the embodiment and the processing power required by the entity. For example, the stool evaluation tool 106 can run in a single computer 400 or multiple computers 400 communicating with each other through a network such as in a server farm.

The computers 400 can lack some of the components described above, such as graphics adapters 412, and displays 418.

### V. Systems

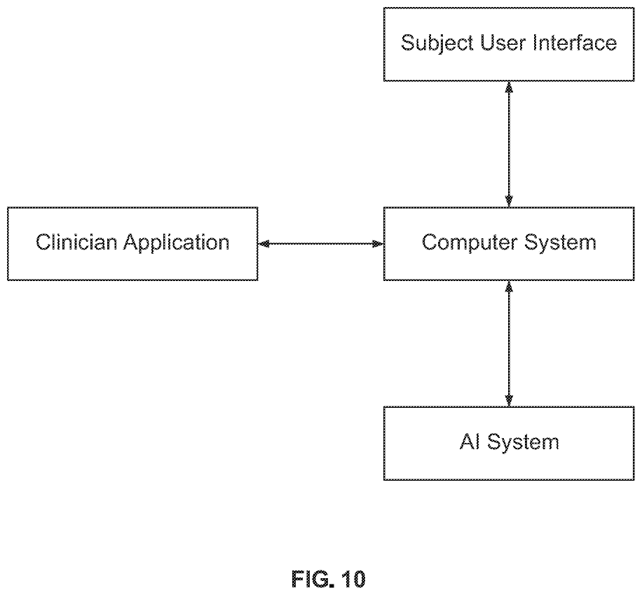

Further disclosed herein are systems for implementing one or more AI engines for determining a stool condition. In various embodiments, such a system can include at least the stool evaluation tool 106 described above in FIGS. 1-2. In some embodiments, the stool evaluation tool 106 is embodied as a computer system, such as a computer system with example computer 400 described in FIG. 4. As depicted in FIG. 10, in some embodiments, the computer system is operatively communicated with a user interface (e.g., for display and receiving input), an AI system (as described herein), and/or a clinician application or computer system (e.g., a healthcare administrator), as described herein.

### VI. Examples

### Example 1: Comparison of Stool Condition Determination Between a Self-Assessment by a Subject and Using Artificial Intelligence

Subjects with diarrhea-predominant irritable bowel syndrome captured images of stool for 2 weeks, wherein a stool evaluation tool performed a stool assessment for each stool, determining a stool condition based on characteristics i) shape and texture, ii) consistency, iii) fragmentation, iv) edge fuzziness, and v) volume. For this experiment, the shape and texture used the Bristol Stool Scale. In the validation phase, using two expert gastroenterologists as a gold standard, sensitivity, specificity, accuracy and diagnostic odds ratios of subject-reported vs AI-graded Bristol Stool Scale scores were compared. Bristol Stool Scale scores were reported by the AI (stool evaluation tool) and subject self-assessed scores. During an implementation phase, the subject Bristol Stool Scale scores and the AI stool characteristics scores (e.g., based on the stool assessment) were correlated with diarrhea-predominant irritable bowel syndrome symptom severity scores.

During validation-phase, there was good agreement between the two experts and AI characterizations for the stool characteristics (intraclass correlation coefficients [ICC]=0.782-0.852), stool consistency (ICC=0.873-0.890), edge fuzziness (ICC=0.836-0.839), fragmentation (ICC=0.837-0.863), and volume (ICC=0.725-0.851). The AI outperformed subjects' self-reports in categorizing daily average Bristol Stool Scale scores as constipation, normal, or diarrhea. In implementation-phase (n=25), agreement between AI and self-reported BSS scores was moderate (ICC=0.61). AI stool characterization also correlated better than subject reports with diarrhea severity scores.

Accordingly, the stool evaluation tool, using AI is capable of determining Bristol Stool Scale score and other stool characteristics with high accuracy when compared with two expert gastroenterologists. Moreover, trained AI was superior to subject self-reporting of the Bristol Stool Scales.

### Example 2: Evaluation of Efficacy of a Medication with Respect to Stool Condition

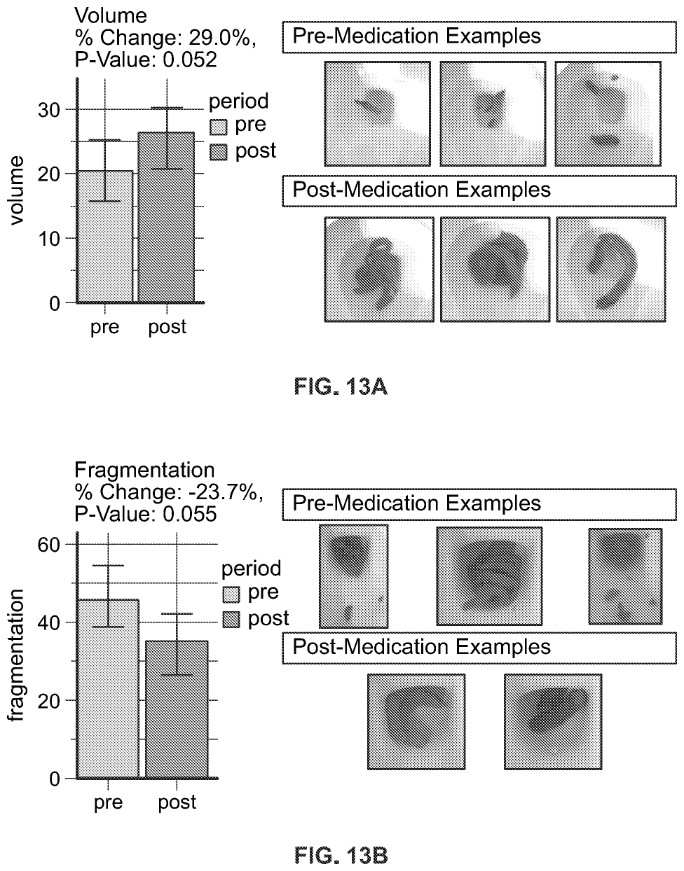

A clinical trial was performed to determine an effectiveness of a medication on improving the stool condition for a subject. The stool condition for the subject was determined and tracked for two weeks prior to taking the medication, using the stool evaluation tool. The stool condition was then determined for two weeks after taking the medication. The stool condition was shown to improve after the monitoring period. The volume for example, increased by nearly 30%, where pre-medication the amount of stool was low (see FIG. 13A). The fragmentation also was reduced in the stool after receiving the medication, also an indication of improved stool condition (see FIG. 13B).

All publications, patents, patent applications and other documents cited in this application are hereby incorporated by reference herein in their entireties for all purposes to the same extent as if each individual publication, patent, patent application or other document were individually indicated to be incorporated by reference for all purposes. While various specific embodiments have been illustrated and described, the above specification is not restrictive. It will be appreciated that various changes can be made without departing from the spirit and scope of the present disclosure(s). Many variations will become apparent to those skilled in the art upon review of this specification.

1. A non-transitory computer readable medium for determining a stool condition for a subject, the non-transitory computer readable medium comprising instructions that, when executed by a processor, cause the processor to perform operations including:
a. receiving an image of stool corresponding to a bowel movement;
b. determining a plurality of characteristics associated with the stool based on the image; and
c. performing a stool assessment based on the plurality of characteristics, the stool assessment correlating with the stool condition;
wherein the plurality of characteristics comprises one or more of a shape and texture, consistency, fragmentation, fuzziness, and volume.

2. The non-transitory computer readable medium of claim 1, wherein the stool condition is based on a plurality of images of stools corresponding to a plurality of bowel movements, wherein a stool assessment is performed for each image of stool.

3. The non-transitory computer readable medium of claim 1 or 2, wherein performing the stool assessment further comprises identifying one or more medical conditions, illnesses, and/or diseases for the subject.

4. The non-transitory computer readable medium of any one of claims 1 to 3, wherein the one or more medical conditions, illnesses, and/or diseases comprises Irritable Bowel Syndrome, Crohn's Disease, Ulcerative Colitis, Hepatic Encephalopathy, or a combination thereof.

5. The non-transitory computer readable medium of any one of claims 1 to 4, wherein the operations further include identifying one or more correlations between one or more subject conditions and the stool condition.

6. The non-transitory computer readable medium of claim 5, wherein the one or more subject conditions comprises a diet intake, one or more lifestyle conditions, one or more medications, or a combination thereof.

7. The non-transitory computer readable medium of any one of claims 2 to 6, wherein the operations further includes determining an effectiveness of a medication based on a change in the stool condition between one or more bowel movements.

8. The non-transitory computer readable medium of any one of claims 1 to 7, wherein the operations further includes providing an intervention recommendation based on the stool condition.

9. The non-transitory computer readable medium of claim 8, wherein the recommendation comprises a change to i) one or more of the subject's diet, ii) one or more lifestyle conditions, and/or iii) one or more medications being received by the subject.

10. The non-transitory computer readable medium any one of claims 1 to 9, wherein determining the plurality of characteristics and/or performing the stool assessment comprises using a machine learning algorithm.

11. The non-transitory computer readable medium of claim 10, wherein the machine learning algorithm uses a trained data set in operative communication with the processor to determine the plurality of characteristics and/or perform the stool assessment.

12. The non-transitory computer readable medium of claim 11, wherein the trained data set comprises a plurality of past images of stool correlated with a plurality of characteristics.

13. The non-transitory computer readable medium any one of claims 1 to 12, wherein the processor is a part of computing device.

14. The non-transitory computer readable medium of claim 13, wherein the computing device comprises a mobile device, a desktop, a laptop, and/or a remote computing server.

15. The non-transitory computer readable medium of claim 14, wherein the mobile device comprises a smart phone, a tablet, a smartwatch, or any combination thereof.

16. The non-transitory computer readable medium of any one of claims 1 to 15, wherein receiving the image of the stool comprises using a camera in operative communication with the processor and configured to capture the image.

17. The non-transitory computer readable medium of any one of claims 13 to 16, wherein the computing device comprises said camera.

18. The non-transitory computer readable medium of any one of claims 13 to 17, wherein the computing device is in operative communication with a display to output the stool assessment.

19. The non-transitory computer readable medium of claim 18, wherein the display is in operative communication with the camera, such that the display provides guiding features to capture the image.

20. The non-transitory computer readable medium of claim 19, wherein the guiding features comprises a shape of a toilet seat defining a central area, such that the image of the stool is located within the central area when the image is captured.

21. The non-transitory computer readable medium of any one of claims 1 to 20, wherein the stool assessment comprises a score and/or rating relating to each characteristic of the plurality of characteristics.

22. The non-transitory computer readable medium of any one of claims 1 to 21, wherein the plurality of characteristics comprises consistency, wherein the corresponding score and/or rating corresponds to a liquid to solid scale of the stool, wherein one end of the scale corresponds to a fully liquid stool, and another end of the scale corresponds to a fully solid stool.

23. The non-transitory computer readable medium of any one of claims 1 to 22, wherein the plurality of characteristics comprises fragmentation, wherein the corresponding score and/or rating corresponds to a degree relating to a number of pieces present in the stool, wherein one end of the scale corresponds to a single stool piece, and another end of the scale corresponds to a large number of stool pieces.

24. The non-transitory computer readable medium of any one of claims 1 to 23, wherein the plurality of characteristics comprises fuzziness, wherein the corresponding score and/or rating corresponds to a degree of a clear boundary existing between the stool and a background in the image, wherein one end of the scale corresponds to a clear distinguishable or substantially distinguishable boundary, and another end of the scale corresponds to an indistinguishable or substantially indistinguishable boundary.

25. The non-transitory computer readable medium of any one of claims 1 to 24, wherein the plurality of characteristics comprises volume, wherein the corresponding score and/or rating corresponds to a size of the stool, wherein one end of the scale corresponds to a small size, and another end of the scale corresponds to a large size stool.

26. The non-transitory computer readable medium any one of claims 1 to 25, wherein the operations further include i) sending to the stool assessment to a healthcare provider, and/or ii) receiving input from the healthcare provider.

27. The non-transitory computer readable medium of claim 26, wherein the processor is in operative communication with the healthcare provider via a communication module.

28. The non-transitory computer readable medium of any one of claims 1 to 27, wherein obtaining an image comprises obtaining a plurality of images of the stool, wherein determining the plurality of characteristics and outputting the stool assessment is based on the plurality of images.

29. The non-transitory computer readable medium of any one of claims 1 to 28, wherein the operations further comprises validating the stool assessment performed based on comparing a score for one or more of the plurality of characteristics between i) the image and one or more other images of the stool, and/or ii) the stool assessment and one or more other stool assessments performed for the image.

30. A method for determining a stool condition for a subject, the method comprising:
a. receiving an image of stool corresponding to a bowel movement;
b. determining a plurality of characteristics associated with the stool based on the image; and
c. performing a stool assessment based on the plurality of characteristics, the stool assessment correlating with the stool condition;
wherein the plurality of characteristics comprises one or more of a shape and texture, consistency, fragmentation, fuzziness, and volume.

31. The method of claim 30, wherein the stool condition is based on a plurality of images of stools corresponding to a plurality of bowel movements, wherein a stool assessment is performed for each image of stool.

32. The method of claim 30 or 31, wherein performing the stool assessment further comprises identifying one or more medical conditions, illnesses, and/or diseases for the subject.

33. The method of any one of claims 30 to 32, wherein the one or more medical conditions, illnesses, and/or diseases comprises Irritable Bowel Syndrome, Crohn's Disease, Ulcerative Colitis, Hepatic Encephalopathy, or a combination thereof.

34. The method of any one of claims 30 to 33, further comprising identifying one or more correlations between one or more subject conditions and the stool condition.

35. The method of claim 34, wherein the one or more subject conditions comprises a diet intake, one or more lifestyle conditions, one or more medications, or a combination thereof.

36. The method of any one of claims 31 to 35, further comprising determining an effectiveness of a medication based on a change in the stool condition between one or more bowel movements.

37. The method of any one of claims 30 to 36, further comprising providing an intervention recommendation based on the stool condition.

38. The method of claim 37, wherein the recommendation comprises a change to i) one or more of the subject's diet, ii) one or more lifestyle conditions, and/or iii) one or more medications being received by the subject.

39. The method any one of claims 30 to 38, wherein determining the plurality of characteristics and/or performing the stool assessment comprises using a machine learning algorithm.

40. The method of claim 39, wherein the machine learning algorithm uses a trained data set in operative communication with the processor to determine the plurality of characteristics and/or perform the stool assessment.

41. The method of claim 40, wherein the trained data set comprises a plurality of past images of stool correlated with a plurality of characteristics.

42. The method any one of claims 30 to 41, wherein the processor is a part of computing device.

43. The method of claim 42, wherein the computing device comprises a mobile device, a desktop, a laptop, and/or a remote computing server.

44. The method of claim 43, wherein the mobile device comprises a smart phone, a tablet, a smartwatch, or any combination thereof.

45. The method of any one of claims 30 to 44, wherein receiving the image of the stool comprises using a camera in operative communication with the processor and configured to capture the image.

46. The method of any one of claims 42 to 45, wherein the computing device comprises said camera.

47. The method of any one of claims 42 to 46, wherein the computing device is in operative communication with a display to output the stool assessment.

48. The method of claim 47, wherein the display is in operative communication with the camera, such that the display provides guiding features to capture the image.

49. The method of claim 48, wherein the guiding features comprises a shape of a toilet seat defining a central area, such that the image of the stool is located within the central area when the image is captured.

50. The method of any one of claims 30 to 49, wherein the stool assessment comprises a score and/or rating relating to each characteristic of the plurality of characteristics.

51. The method of any one of claims 30 to 50, wherein the plurality of characteristics comprises consistency, wherein the corresponding score and/or rating corresponds to a liquid to solid scale of the stool, wherein one end of the scale corresponds to a fully liquid stool, and another end of the scale corresponds to a fully solid stool.

52. The method of any one of claims 30 to 51, wherein the plurality of characteristics comprises fragmentation, wherein the corresponding score and/or rating corresponds to a degree relating to a number of pieces present in the stool, wherein one end of the scale corresponds to a single stool piece, and another end of the scale corresponds to a large number of stool pieces.

53. The method of any one of claims 30 to 52, wherein the plurality of characteristics comprises fuzziness, wherein the corresponding score and/or rating corresponds to a degree of a clear boundary existing between the stool and a background in the image, wherein one end of the scale corresponds to a clear distinguishable or substantially distinguishable boundary, and another end of the scale corresponds to an indistinguishable or substantially indistinguishable boundary.

54. The method of any one of claims 30 to 53, wherein the plurality of characteristics comprises volume, wherein the corresponding score and/or rating corresponds to a size of the stool, wherein one end of the scale corresponds to a small size, and another end of the scale corresponds to a large size stool.

55. The method any one of claims 30 to 54, further comprising i) sending to the stool assessment to a healthcare provider, and/or ii) receiving input from the healthcare provider.

56. The method of claim 55, wherein the processor is in operative communication with the healthcare provider via a communication module.

57. The method of any one of claims 30 to 56, wherein obtaining an image comprises obtaining a plurality of images of the stool, wherein determining the plurality of characteristics and outputting the stool assessment is based on the plurality of images.

58. The method of any one of claims 30 to 57, further comprising validating the stool assessment performed based on comparing a score for one or more of the plurality of characteristics between i) the image and one or more other images of the stool, and/or ii) the stool assessment and one or more other stool assessments performed for the image.

59. A system for determining a stool condition for a subject, the system comprising:
one or more processors; and
one or more memories storing instructions that, when executed by the one or more processors, cause the system to perform operations including:
a. receiving an image of stool corresponding to a bowel movement;
b. determining a plurality of characteristics associated with the stool based on the image; and
c. performing a stool assessment based on the plurality of characteristics, the stool assessment correlating with the stool condition;
wherein the plurality of characteristics comprises one or more of a shape and texture, consistency, fragmentation, fuzziness, and volume.

60. The system of claim 59, wherein the stool condition is based on a plurality of images of stools corresponding to a plurality of bowel movements, wherein a stool assessment is performed for each image of stool.

61. The system of claim 59 or 60, wherein performing the stool assessment further comprises identifying one or more medical conditions, illnesses, and/or diseases for the subject.

62. The system of any one of claims 59 to 61 wherein the one or more medical conditions, illnesses, and/or diseases comprises Irritable Bowel Syndrome, Crohn's Disease, Ulcerative Colitis, Hepatic Encephalopathy, or a combination thereof.

63. The system of any one of claims 59 to 62, wherein the operations further include identifying one or more correlations between one or more subject conditions and the stool condition.

64. The system of claim 63, wherein the one or more subject conditions comprises a diet intake, one or more lifestyle conditions, one or more medications, or a combination thereof.

65. The system of any one of claims 60 to 64, wherein the operations further includes determining an effectiveness of a medication based on a change in the stool condition between one or more bowel movements.

66. The system of any one of claims 59 to 65, wherein the operations further includes providing an intervention recommendation based on the stool condition.

67. The system of claim 66, wherein the recommendation comprises a change to i) one or more of the subject's diet, ii) one or more lifestyle conditions, and/or iii) one or more medications being received by the subject.

68. The system any one of claims 59 to 67, wherein determining the plurality of characteristics and/or performing the stool assessment comprises using a machine learning algorithm.

69. The system of claim 68, wherein the machine learning algorithm uses a trained data set in operative communication with the processor to determine the plurality of characteristics and/or perform the stool assessment.

70. The system of claim 69, wherein the trained data set comprises a plurality of past images of stool correlated with a plurality of characteristics.

71. The system any one of claims 59 to 70, wherein the processor is a part of computing device.

72. The system of claim 71, wherein the computing device comprises a mobile device, a desktop, a laptop, and/or a remote computing server.

73. The system of claim 72, wherein the mobile device comprises a smart phone, a tablet, a smartwatch, or any combination thereof.

74. The system of any one of claims 59 to 73, wherein receiving the image of the stool comprises using a camera in operative communication with the processor and configured to capture the image.

75. The system of any one of claims 71 to 74, wherein the computing device comprises said camera.

76. The system of any one of claims 71 to 75, wherein the computing device is in operative communication with a display to output the stool assessment.

77. The system of claim 76, wherein the display is in operative communication with the camera, such that the display provides guiding features to capture the image.

78. The system of claim 77, wherein the guiding features comprises a shape of a toilet seat defining a central area, such that the image of the stool is located within the central area when the image is captured.

79. The system of any one of claims 59 to 78, wherein the stool assessment comprises a score and/or rating relating to each characteristic of the plurality of characteristics.

80. The system of any one of claims 59 to 79, wherein the plurality of characteristics comprises consistency, wherein the corresponding score and/or rating corresponds to a liquid to solid scale of the stool, wherein one end of the scale corresponds to a fully liquid stool, and another end of the scale corresponds to a fully solid stool.

81. The system of any one of claims 59 to 80, wherein the plurality of characteristics comprises fragmentation, wherein the corresponding score and/or rating corresponds to a degree relating to a number of pieces present in the stool, wherein one end of the scale corresponds to a single stool piece, and another end of the scale corresponds to a large number of stool pieces.

82. The system of any one of claims 59 to 81, wherein the plurality of characteristics comprises fuzziness, wherein the corresponding score and/or rating corresponds to a degree of a clear boundary existing between the stool and a background in the image, wherein one end of the scale corresponds to a clear distinguishable or substantially distinguishable boundary, and another end of the scale corresponds to an indistinguishable or substantially indistinguishable boundary.

83. The system of any one of claims 59 to 82, wherein the plurality of characteristics comprises volume, wherein the corresponding score and/or rating corresponds to a size of the stool, wherein one end of the scale corresponds to a small size, and another end of the scale corresponds to a large size stool.

84. The system any one of claims 59 to 83, wherein the operations further include i) sending to the stool assessment to a healthcare provider, and/or ii) receiving input from the healthcare provider.

85. The system of claim 84, wherein the processor is in operative communication with the healthcare provider via a communication module.

86. The system of any one of claims 59 to 85, wherein obtaining an image comprises obtaining a plurality of images of the stool, wherein determining the plurality of characteristics and outputting the stool assessment is based on the plurality of images.

87. The system of any one of claims 59 to 86, wherein the operations further comprises validating the stool assessment performed based on comparing a score for one or more of the plurality of characteristics between i) the image and one or more other images of the stool, and/or ii) the stool assessment and one or more other stool assessments performed for the image.
